(19) United States
(12) Patent Application Publication
(10) Pub. No.: US 2003/0206934 A1
(43) Pub. Date: Nov. 6, 2003

(54) Low-lipid cosmetic and dermatological preparations in the form of O/W emulsions containing fatty acids

(75) Inventors: Heidi Riedel, Hamburg (DE); Wiebke Lindemann, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/383,899

(22) Filed: Mar. 7, 2003

Related U.S. Application Data
(63) Continuation of application No. PCT/EP01/09306, filed on Aug. 11, 2001.

(30) Foreign Application Priority Data
Sep. 7, 2000 (DE) 100 44 313.3

(51) Int. Cl. A61K 7/06; A61K 7/11

(52) U.S. Cl. 424/401; 424/70.12

(57) ABSTRACT

Cosmetic or dermatological preparations in the form of an O/W emulsion comprising

(I) 1-5% by weight, based on the total weight of the preparations, of one or more neutralized or partially neutralized C₁₄-C₃₂-fatty acids

(II) where sodium hydroxide and/or potassium hydroxide are chosen as neutralizing base, corresponding to a pH range of the preparations of 6.0-8.0 in neutralized or partially neutralized form,

(III) where the content of alkylated ammonium bases is chosen to be less than 0.01% by weight

(IV) 0.1 to 1.5% by weight, based on the total weight of the preparations, of one or more mono- and/or diesters of glycerol and/or of propylene glycol and/or of glycol,

(V) 0.5 to 3% by weight, based on the total weight of the preparations, of one or more fatty alcohols chosen from the group of branched and unbranched alkyl alcohols having 12 to 40 carbon atoms,

(VI) 1 to 9% by weight, based on the total weight of the preparations, of a lipid phase composed of

(a) one or more lipids with a melting point below 30° C.

(b) one or more lipids with a melting point above 30° C.

(VII) where the ratio of (a) and (b) is in the range from 2:1 to 6:1.

(VIII) where lipids according to (a) comprise 0-5% of silicone oil, based on the total weight of the preparations

where the ratio of lipids (a): silicone oils (VIII) is preferably in the range from 5:1 to 1:2.

### CROSS-REFERENCE TO RELATED APPLICATIONS

This is a continuation application of PCT/EP01/09306, filed Aug. 11, 2001, which is incorporated herein by reference in its entirety, and also claims the benefit of German Priority Application No. 100 44 313.3, filed Sep. 7, 2000.

### FIELD OF THE INVENTION

The present invention relates to cosmetic and dermatological emulsions, in particular skincare cosmetic and dermatological emulsions. In an advantageous embodiment, the present invention relates to an application that allows an increase in the stability of preparations, in particular emulsions, preferably ONV emulsions, which contain fatty acids.

### BACKGROUND OF THE INVENTION

The skin is the largest human organ. Amongst its many functions (for example for temperature regulation and as a sensory organ) the barrier function, which prevents the skin (and ultimately the entire organism) from drying out, is the most important. At the same time, the skin acts as a protective device against the penetration and absorption of external substances. This barrier function is effected by the epidermis, which, as the outermost layer, forms the actual protective sheath against the environment. Providing about one tenth of the total thickness, it is also the thinnest layer of the skin.

The epidermis is a stratified tissue in which the outer layer, the horny layer (Stratum corneum), is the part which is of significance for the barrier function. The Elias skin model, which is currently recognized in the specialist field (P. M. Elias, Structure and Function of the Stratum Corneum Permeability Barrier, Drug Dev. Res. 13, 1988, 97-105), describes the horny layer as a two-component system, similar to a brick wall (bricks and mortar model). In this model, the horny cells (corneocytes) correspond to the bricks, and the lipid membrane in the intercellular spaces, which is of complex composition, corresponds to the mortar. This system is essentially a physical barrier to hydrophilic substances, but, because of its narrow and multilayered structure, can equally also be passed by lipophilic substances only with difficulty.

Apart from their barrier action against external chemical and physical influences, the epidermal lipids also contribute to the holding together of the horny layer and have an effect on the smoothness of the skin. In contrast to the sebaceous gland lipids, which do not form a continuous film on the skin, the epidermal lipids are distributed over the entire horny layer.

The extremely complex interaction of the moisture-binding substances and of the lipids of the upper layers of the skin is very important for the regulation of skin moisture. For this reason, cosmetics generally comprise, in addition to balanced lipid mixtures and water, water-binding substances.

As well as the chemical composition, however, the physical behavour of these substances is also of importance. The development of very biocompatible emulsifiers and surfactants is therefore desirable. Products formulated therewith aid the liquid-crystalline organization of the intercellular lipids of the Stratum corneum, thereby improving the barrier properties of the horny layer. It is particularly advantageous if their molecular constituents consist of substances which are naturally occurring in the epidermis.

Cosmetic skin care primarily means that the natural function of the skin as a barrier against environmental influences (e.g. dirt, chemicals, microorganisms) and against the loss of endogenous substances (e.g. water, natural fats, electrolytes) is strengthened or restored.

If this function is impaired, increased resorption of toxic or allergenic substances or attack by microorganisms may result, leading to toxic or allergic skin reactions.

Another aim of skin care is to compensate for the loss by the skin of lipids and water caused by daily washing. This is particularly important when the natural regeneration ability is insufficient. Furthermore, skincare products should protect against environmental influences, in particular against sun and wind, and delay skin aging.

Medicinal topical compositions generally comprise one or more medicaments in an effective concentration. For the sake of simplicity, in order to distinguish clearly between cosmetic and medicinal use and corresponding products, reference is made to the legal provisions in the Federal Republic of Germany (e.g. Cosmetics Directive, Foods and Drugs Act).

Customary cosmetic forms of application are emulsions. This term generally means a heterogeneous system of two liquids which are immiscible or miscible only to a limited extent with one another, which are usually referred to as phases. One is in the form of droplets (disperse or internal phase), whilst the other liquid forms a continuous (coherent or internal) phase. Less common forms of application are multiple emulsions, i.e. those which, in the droplets of the dispersed (or discontinuous) phase, comprise for their part droplets of a further dispersed phase, e.g. W/O/W emulsions and O/W/O emulsions.

More recent findings have recently led to a better understanding of cosmetic emulsions which are of relevance in practice. Here, it is assumed that the emulsifier mixtures used in excess form lamellar liquid-crystalline phases or crystalline gel phases. In the gel network theory, stability and physicochemical properties of such emulsions are attributed to the formation of viscoelastic gel networks.

In order to be able to ensure the metastability of emulsions, interface-active substances, i.e. emulsifiers, are usually necessary. The use per se of customary cosmetic emulsifiers is entirely acceptable. Nevertheless, emulsifiers, as ultimately any chemical substance, may in certain cases cause allergic reactions or reactions based on oversensitivity of the user. For example, it is known that in some particularly sensitive people, certain light dermatoses are triggered by certain emulsifiers and simultaneous action of sunlight.

It is possible to prepare emulsifier-free preparations which, for example, have, in an aqueous phase, dispersed oil droplets, similar to an O/w emulsion. A prerequisite for this may be that the continuous aqueous phase has a gel framework which stabilizes the dispersed phase, and other conditions besides. Such systems are sometimes called hydrodispersions or oleodispersions depending on which is the disperse phase and which is the continuous phase.

For cosmetics technology, it is neither necessary nor possible to dispense with emulsifiers altogether, especially since there is a certain choice of particularly mild emulsifiers. However, the prior art lacks a satisfactorily broad range of such emulsifiers which would then also significantly broaden the application spectrum of correspondingly mild cosmetic preparations which are tolerated by the skin.

Physicochemically stable and sensorily high-value emulsions with a low lipid content are difficult to formulate and prepare industrially. This is true in particular for stearate systems, which comprise exclusively sodium hydroxide or else potassium hydroxide as neutralizing agents.

Known cosmetic preparations are so-called stearate emulsions, i.e. those in which stearic acid and/or palmitic acid or alkali metal salts of stearic acid and/or of palmitic acid are effective as emulsifier. These preparations can advantageously be in the form of O/W emulsions and are characterized by a good feel on the skin. A disadvantage, however, is that fatty acids in a pH range from 3.5-8.0 have a tendency toward crystallization, as a result of which the pleasant feel on the skin and the external appearance of a corresponding preparation is severely impaired.

### SUMMARY OF THE INVENTION

The object was therefore to remedy all of these the disadvantages of the prior art. In particular, the intention was to provide products having reduced stickiness or greasiness. Products in the field of care cosmetics, decorative cosmetics and pharmacological technology should likewise be freed from the described disadvantages of the prior art.

Furthermore, it was an object of the invention to develop cosmetic bases for cosmetic preparations which are characterized by good skin compatibility.

Furthermore, it was an object of the present invention to provide products with as broad an application diversity as possible. For example, the intention was to provide bases for preparation forms such as cleansing emulsions, face and bodycare preparations, and also distinctly medicinal-pharmaceutical administration forms, for example preparations for treating acne and other skin conditions.

Surprisingly, it has been found, and herein lies the solution to these problems, that cosmetic and dermatological preparations in the form of O/W emulsions, comprising

(I) 1-5% by weight, based on the total weight of the preparations, of one or more neutralized or partially neutralized C₁₄-C₃₂-fatty acids

(II) where sodium hydroxide and/or potassium hydroxide are chosen as neutralizing base, corresponding to a pH range of the preparations of 6.0-8.0 in neutralized or partially neutralized form,

(III) where the content of alkylated ammonium bases is chosen to be less than 0.01% by weight

(IV) 0.1 to 1.5% by weight, based on the total weight of the preparations, of one or more mono- and/or diesters of glycerol and/or of propylene glycol and/or of glycol,

(V) 0.5 to 3% by weight, based on the total weight of the preparations, of one or more fatty alcohols chosen from the group of branched and unbranched alkyl alcohols having 12 to 40 carbon atoms,

(VI) 1 to 9% by weight, based on the total weight of the preparations, of a lipid phase composed of

(a) one or more lipids with a melting point below 30° C.

(b) one or more lipids with a melting point above 30° C.

(VII) where the ratio of (a) and (b) is in the range from 2:1 to 6:1.

(VIII) where lipids according to (a) comprise 0-5% of silicone oil, based on the total weight of the preparations

(IX) where the ratio of lipids (a): silicone oils (VIII) is preferably in the range from 5:1 to 1:2,

overcome the disadvantages of the prior art.

It was therefore not foreseeable by the person skilled in the art that the preparations according to the invention:

are more effective moisture-donating preparations,

better promote skin smoothing,

are characterized by better care action,

are better vehicles for cosmetic and medicinal-dermatological active ingredients,

have higher stability to physical decomposition, in particular phase separation,

have higher stability to crystallization of the fatty acids used,

would be characterized by better biocompatibility, and

would be characterized by better feel on the skin and by higher cosmetic elegance than the prior art preparations. According to the invention, it is possible to achieve very good physicochemical stabilities without instabilities after a prolonged storage period, in particular also at high temperatures (e.g. 6 months at 40° C., 2 months at 50° C., 1 week at 70° C.). Consequently, it is possible to prepare formulations with above-average skincare and excellent, balanced sensory properties. This is characterized in particular by a good absorption capacity and by a notably low residue, even though the product conveys a soft richness during spreading.

The present invention relates, in a particular embodiment, to cosmetic or pharmaceutical preparations having a reduced feel of stickiness, to processes for their preparation, and to the use of active ingredients for reducing the feel of stickiness of cosmetic preparations.

### DETAILED DESCRIPTION OF THE INVENTION

The preparations according to the invention can be formulated both in flowable form and also in cream form, have very good cosmetic properties, in particular with regard to stickiness, and have very good skin compatibility and skincare performance.

Preferred fatty acids are stearic acid and/or palmitic acid (stearin).

Advantageous embodiments of the present invention relate to cosmetic and dermatological preparations comprising

(IV) 0.1 to 1.5% by weight, based on the total weight of the preparations, chosen from the group consisting of glyceryl monostearate, glyceryl distearate, propylene glycol monostearate, glyceryl isostearate, glyceryl lanolate, glyceryl myristate, glyceryl laurate, glyceryl oleate, glyceryl stearate citrate.

For the purposes of the present disclosure, the general term for fats, oils, waxes and the like which is sometimes used is the expression “lipids”, with which the person skilled in the art is entirely familiar. The terms “oily phase” and “lipid phase” are also used synonymously.

The fatty alcohol(s) is/are advantageously chosen from the group of monohydric alcohols having 12-30 carbon atoms in straight chains, dodecanol (lauryl alcohol, C₁₂H₂₅OH), (myristyl alcohol, C₁₄H₂₉OH), hexadecanol (cetyl alcohol, C₁₆H₃₃OH), octadecanol (stearyl alcohol, C₁₈H₃₇OH), oleyl alcohol (C₁₈H₃₅OH), eicosyl alcohol (C₂₀H₃₉OH), docosanyl alcohol (C₂₂H₄₃OH), tetracosanyl alcohol (carnaubyl alcohol, C₂₄H₄₉OH), hexacosanyl alcohol (ceryl alcohol, C₂₆H₅₃OH), triacontyl alcohol (myricyl alcohol, C₃₀H₆₁OH).

It is preferred to choose the proportion of the silicone oils of the preparations according to the invention from the group of cyclic and/or linear silicones, which are also referred to as “silicone oils” within the scope of the present disclosure. Such silicones or silicone oils can be in the form of monomers, which are generally characterized by structural elements, as follows:

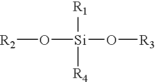

Linear silicones having two or more siloxyl units which are to be used advantageously according to the invention are generally characterized by structural elements as follows:

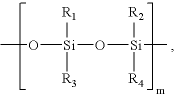

where the silicon atoms can be substituted by identical or different alkyl radicals and/or aryl radicals, which are shown here by the radicals R₁-R₄ as a generalization (that is to say that the number of different radicals is not necessarily limited to 4). m can assume values from 2-200 000.

Cyclic silicones which are to be used advantageously according to the invention are generally characterized by structural elements, as follows

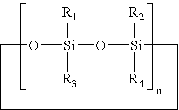

where the silicon atoms can be substituted by identical or different alkyl radicals and/or aryl radicals, which are shown here by the radicals R₁-R₄ as a generalization (that is to say that the number of different radicals is not necessarily limited to 4). n can assume values from 3/2 to 20. Fractions for n allow for the fact that odd numbers of siloxyl groups may be present in the cycle.

Phenyltrimethicone is advantageously chosen as the silicone oil. Other silicone oils, for example, dimethicone, phenyldimethicone, cyclomethicone (octamethylcyclotetrasiloxane), hexamethylcyclotrisiloxane, polydimethylsiloxane, poly(methylphenylsiloxane), cetyldimethicone, and behenoxydimethicone can also be used advantageously for the purposes of the present invention.

Mixtures of cyclomethicone and isotridecyl isononanoate, and those of cyclomethicone and 2-ethylhexyl isostearate are also advantageous.

Other oil components can, for example, be chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, and from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, such as, for example, jojoba oil.

In addition, the oil phase can advantageously be chosen from the group of dialkyl ethers, and the group of saturated or unsaturated, branched or unbranched alcohols. It is particularly advantageous if the oil phase of the W/O emulsions according to the invention has a content of C₁₂-C₁₅ alkyl benzoate or cetylstearyl ethylhexanoate, or consists entirely of this.

Any mixtures of such oil and wax components can also be used advantageously for the purposes of the present invention. In some instances, it may also be advantageous to use waxes, for example cetyl palmitate, as the sole lipid component of the oil phase.

Advantageous oil components are also, for example, those chosen from the group of branched and unbranched hydrocarbons and hydrocarbon waxes, in particular Vaseline (petrolatum), paraffin oil, squalane and squalene, polyolefins and hydrogenated polyisobutenes. Of the polyolefins, polydecenes are the preferred substances. Table 1 below lists lipids which are advantageous according to the invention as individual substances or else in a mixture with one another. The respective interfacial tensions toward water are given in the last column. It is, however, also advantageous to use mixtures of more or less polar [lacuna] and the like.

The oil phase can also be chosen advantageously from the group of branched and unbranched hydrocarbons and hydrocarbon waxes, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24, in particular 12-18, carbon atoms. The fatty acid triglycerides can, for example, be advantageously chosen from the group of synthetic, semisynthetic and natural oils, e.g. olive oil, sunflower oil, soya oil, peanut oil, rapeseed oil, almond oil, palm oil, coconut oil, palm kernel oil and the like.

Lipids with a melting point above 30° C. to be used advantageously according to the invention can be chosen from the group of vegetable waxes, animal waxes, mineral waxes and petrochemical waxes. Examples which are favourable according to the invention are candelilla wax, carnauba wax, japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microcrystalline waxes, provided the conditions required in the main claim are observed.

Further advantageous lipids with a melting point above 30° C. are chemically modified waxes and synthetic waxes, such as, for example, those obtainable under the trade names Syncrowax HRC (glyceryl tribehenate), Syncrowax HGLC (C₁₆₋₃₆ fatty acid triglyceride) and Syncrowax AW 1C (C₁₈₋₃₆ fatty acid) from CRODA GmbH, and montan ester waxes, Sasol waxes, hydrogenated jojoba waxes, synthetic or modified beeswaxes (e.g. dimethicone copolyol beeswax and/or C₃₀₋₅₀ alkyl beeswax), polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, trihydroxystearin, fatty acids, fatty acid esters, and glycol esters, such as, for example, C₂₀₋₄₀ alkyl stearate, C₂₀₋₄₀ alkylhydroxystearoyl stearate and/or glycol montanate. Also advantageous are certain organosilicon compounds, which have similar physical properties to the specified fat and/or wax components, such as, for example, stearoxytrimethylsilane, provided the conditions required in the main claim are observed.

According to the invention, the lipids having a melting point above 30° C. can be present either individually or as a mixture.

Any desired mixtures of such oil and wax components can also be used advantageously for the purposes of the present invention.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅ alkyl benzoate, cetylstearyl ethylhexanoate, isopropyl myristate, caprylic/capric triglyceride, dicaprylyl ether, provided the conditions required in the main claim are observed.

Mixtures of C₁₂₋₁₅ alkyl benzoate and 2-ethylhexyl isostearate, cetearyl ethylhexanoates, mixtures of cetylstearyl ethylhexanoate and isopropyl myristate, and mixtures of C₁₂₋₁₅ alkyl benzoate, 2-ethylhexyl isostearate and isotridecyl isononanoate are particularly advantageous provided the conditions required in the main claim are observed.

Of the hydrocarbons, paraffin oil, cycloparaffin, squalane, squalene, hydrogenated polyisobutene and polydecene can be used advantageously for the purposes of the present invention provided the conditions required in the main claim are observed.

Basic constituents of the preparations according to the invention which may be used are:

water or aqueous solutions;

aqueous ethanolic solutions;

natural oils and/or chemically modified natural oils and/or synthetic oils;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids; and

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol and, in particular, one or more thickeners which can advantageously be chosen from the group consisting of silicon dioxide, aluminum silicates, polysaccharides or derivatives thereof, e.g. hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination.

For the purposes of the present invention, emulsions according to the invention, for example in the form of a skin protection cream, a skin lotion, a cosmetic milk, for example in the form of a sun protection cream or a sun protection milk, are advantageous and comprise, for example, fats, oils, waxes and/or other fatty substances, and water and one or more emulsifiers as are customarily used for this type of formulation.

The person skilled in the art is of course aware that high-quality cosmetic compositions are in most cases inconceivable without the customary auxiliaries and additives. These include, for example, bodying agents, fillers, perfume, dyes, emulsifiers, additional active ingredients such as vitamins or proteins, light protection agents, stabilizers, insect repellents, alcohols, water, salts, antimicrobials, proteolytic or keratolytic substances, etc.

Corresponding requirements apply mutatis mutandis to the formulation of medicinal preparations.

For the purposes of the present invention, medicinal topical compositions generally comprise one or more medicaments in an effective concentration. For the sake of simplicity, in order to distinguish clearly between cosmetic and medicinal use and corresponding products, reference is made to the legal provisions in the Federal Republic of Germany (for example Cosmetics Directive, Foods and Drugs Act).

Accordingly, for the purposes of the present invention, cosmetic or topical dermatological compositions can, depending on their composition, be used for example as skin protection cream, cleansing milk, sunscreen lotion, nourishing cream, day or night cream, etc. It is in some cases possible and advantageous to use the compositions according to the invention as a base for pharmaceutical formulations.

It is likewise advantageous to make use of the properties according to the invention in the form of decorative cosmetics (make-up formulations).

Those cosmetic and dermatological preparations which are in the form of a sunscreen are also favourable. In addition to the active ingredient used according to the invention, these also preferably comprise in addition at least one UVA filter substance and/or at least one UVB filter substance and/or at least one inorganic pigment.

However, it is also advantageous for the purposes of the present invention to provide cosmetic and dermatological preparations whose main purpose is not protection against sunlight, but which nevertheless contain a content of anti-UV substances. Thus, for example, UV-A and UV-B filter substances are usually incorporated into day creams.

Preparations according to the invention can advantageously comprise substances which absorb UV radiation in the UVB region, the total amount of filter substances being, for example, from 0.1% by weight to 30% by weight, preferably from 0.5 to 10% by weight, in particular from 1 to 6% by weight, based on the total weight of the preparations.

The UVB filters can be oil-soluble or water-soluble. Examples of oil-soluble substances which may be mentioned are:

3-benzylidenecamphor and derivatives thereof, e.g. 3-(4-methylbenzylidene)-camphor,

4-aminobenzoic acid derivatives, preferably 2-ethylhexyl 4-(dimethylamino)benzoate, amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di(2-ethylhexyl) 4-methoxybenzalmalonate; and

2,4,6-trianilino(p-carbo-2′-ethyl-1′-hexyloxy)-1,3,5-triazine.

Advantageous water-soluble substances are:

2-phenylbenzimidazole-5-sulfonic acid and salts thereof, for example sodium, potassium or triethanolammonium salts,

sulfonic acid derivatives of benzophenones, preferably 2-hydroxy-4-methoxybenzophenone-5-sulfonic acid and its salts; and

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts.

The list of specified UVB filters which can be used according to the invention is of course not intended to be limiting.

It can also be advantageous to use UVA filters that are usually present in cosmetic and/or dermatological preparations in preparations according to the invention. Such filter substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. Preparations which comprise these combinations are also provided by the invention. It is possible to use the same amounts of UVA filter substances which were specified for UVB filter substances.

For the purposes of the present invention, cosmetic and/or dermatological preparations can also comprise inorganic pigments which are usually used in cosmetics for protecting the skin against UV radiation. These are oxides of titanium, zinc, iron, zirconium, silicon, manganese, aluminum, cerium and mixtures thereof, and modifications in which the oxides are the active agents. Particular preference is given to pigments based on titanium dioxide. It is possible to use the quantities specified for the above combinations.

The cosmetic and dermatological preparations according to the invention can comprise cosmetic active ingredients, auxiliaries and/or additives as are usually used in such preparations, for example antioxidants, preservatives, bactericides, perfumes, antifoams, dyes, pigments which have a coloring effect, thickeners, surfactants, emulsifiers, emollients, moisturizers and/or humectants, fats, oils, waxes or other usual constituents of a cosmetic or dermatological formulation, such as alcohols, polyols, polymers, foam stabilizers, electrolytes, organic solvents or silicone derivatives.

For the purposes of the present invention, it is advantageous to add other anti-irritant or anti-inflammatory active ingredients to the preparations, in particular batyl alcohol (α-octadecyl glyceryl ether), selachyl alcohol (α-9-octadecenyl glyceryl ether), chimyl alcohol (α-hexadecyl glyceryl ether), bisabolol and/or panthenol.

It is likewise advantageous to add conventional antioxidants to the preparations for the purposes of the present invention. According to the invention, favourable antioxidants can be any antioxidants which are suitable or customary for cosmetic and/or dermatological applications.

The antioxidants are advantageously selected from the group consisting of amino acids (for example glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, ψ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very small tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin, rutinic acid and derivatives thereof, α-glycosylrutin, ferulic acid, furfurylideneglucitol, carnosine, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The amount of antioxidants (one or more compounds) in the preparations is preferably from 0.001 to 30% by weight, particularly preferably 0.05-20% by weight, in particular 1-10% by weight, based on the total weight of the preparation.

If vitamin E and/or derivatives thereof are used as the antioxidant(s), it is advantageous to choose their respective concentrations from the range 0.001-10% by weight, based on the total weight of the formulation.

In some instances, it may also be advantageous to use the preparations according to the invention as a basis for cosmetic or dermatological preparations against hyperpigmentation of the skin (e.g. as so-called skin-whitening products). The customary active ingredients for these purposes, e.g. those based on hydroquinone, kojic acid, ascorbic acid and azelaic acid, and derivatives thereof, can be incorporated into these preparations.

Preparations according to the present invention can also be used as bases for cosmetic or dermatological deodorants or antiperspirants. All active ingredients which are common for deodorants or antiperspirants can be used advantageously, for example odor maskers such as the customary perfume constituents, odor absorbers, for example the phyllosilicates described in laid-open patent specification DE patent 40 09 347, and of these, in particular montmorillonite, kaolinite, ilite, beidellite, nontronite, saponite, hectorite, bentonite, smectite, and also, for example, zinc salts of ricinoleic acid.

Antibacterial agents are likewise suitable for incorporation into the preparations according to the invention. Advantageous substances are, for example, 2,4,4′-trichloro-2′-hydroxydiphenyl ether (Irgasan), 1,6-di(4-chlorophenylbiguanido)hexane (chlorhexidine), 3,4,4′-trichlorocarbanilide, quaternary ammonium compounds, oil of cloves, mint oil, oil of thyme, triethyl citrate, farnesol (3,7,11-trimethyl-2,6,10-dodecatrien-1-ol) and the active ingredients or active ingredient combinations described in laid-open patent specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium hydrogencarbonate can also be used advantageously.

The amount of such active ingredients (one or more compounds) in the preparations according to the invention is preferably from 0.001 to 30% by weight, particularly preferably 0.05-20% by weight, in particular 1-10% by weight, based on the total weight of the preparation.

For the purposes of the present invention, the water phase of the cosmetic preparations can also have gel character and, in addition to an effective content of the substances used according to the invention and the solvents used customarily therefor, preferably water, also comprises other organic thickeners, e.g. gum arabic, xanthan gum, sodium alginate, starch and starch derivatives (e.g. distarch phosphate), cellulose, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates such as, for example, organically modified and also unmodified hectorites, bentonites or the like, or a mixture of polyethylene glycol and polyethylene glycol stearate or distearate. The thickener is present in the gel, for example in an amount between 0.1 and 30% by weight, preferably between 0.5 and 15% by weight.

It can also be advantageous to add interface- or surface-active agents to preparations according to the invention, for example cationic emulsifiers such as, in particular, quaternary surfactants.

Quaternary surfactants contain at least one nitrogen atom which is covalently bonded to 4 alkyl or aryl groups. Irrespective of the pH, this leads to a positive charge. Alkylbetaine, alkylamidopropylbetaine and alkylamidopropylhydroxysulfaine are advantageous. The cationic surfactants used according to the invention can also preferably be chosen from the group of quaternary ammonium compounds, in particular benzyltrialkylammonium chlorides or bromides, such as, for example, benzyldimethylstearylammonium chloride, and also alkyltrialkylammonium salts, for example for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivates and compounds having a cationic character such as amine oxides, for example alkyldimethylamine oxides or alkylaminoethyidimethylamine oxide. In particular, cetyltrimethylammonium salts can be used advantageously.

It is also advantageous to use cationic polymers (e.g. Jaguar® C 162 [hydroxypropyl guar hydroxypropyltrimonium chloride] or modified magnesium aluminum silicates (e.g. quaternium-18 hectorite, which is obtainable, for example, under the trade name Bentone® 38 from Rheox), or stearalkonium hectorite, which is obtainable, for example, under the trade name Softisan® Gel from Hüls AG).

Preparations according to the invention can advantageously also comprise oil thickeners in order to improve the tactile properties of the emulsion and the stick consistency. Advantageous oil thickeners for the purposes of the present invention are, for example, other solids, such as, for example, hydrophobic silicon oxides of the Aerosil® type, which are obtainable from Degussa AG. Advantageous Aerosil® products are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974 and/or Aerosil® R976.

In addition, so-called metal soaps (i.e. the salts of higher fatty acids with the exception of the alkali metal salts) are also advantageous oil thickeners for the purposes of the present invention, such as, for example, aluminum stearate, zinc stearate and/or magnesium stearate.

It is likewise advantageous to add amphoteric or zwitterionic surfactants (e.g. cocoamidopropylbetaine) and moisturizers (e.g. betaine) to preparations according to the invention. Examples of amphoteric surfactants which are to be used advantageously are acyl/dialkylethylenediamine, for example sodium acylamphoacetate, disodium acylamphodipropionate, disodium alkylamphodiacetate, sodium acylamphohydroxypropylsulfonate, disodium acylamphodiacetate and sodium acylamphopropionate, N-alkylamino acids, for example aminopropylalkylglutamide, alkylaminopropionic acid, sodium alkylimidodipropionate and lauroamphocarboxyglycinate.

The amount of interface- or surface-active substances (one or more compounds) in the preparations according to the invention is preferably from 0.001 to 30% by weight, particularly preferably 0.05-20% by weight, in particular 1-10% by weight, based on the total weight of the preparation.

Preparations according to the invention can also comprise active ingredients (one or more compounds) which are chosen from the group: acetylsalicylic acid, atropine, azulene, hydrocortisone and derivatives thereof, e.g. hydrocortisone-17 valerate, vitamins, e.g. ascorbic acid and derivatives thereof, vitamins of the B and D series, very favourably vitamin B₁, vitamin B₁₂ and vitamin D₁, but also bisabolol, unsaturated fatty acids, namely the essential fatty acids (often also called vitamin F), in particular γ-linolenic acid, oleic acid, eicosapentaenoic acid, docosahexaenoic acid and derivatives thereof, chloroamphenicol, caffeine, prostaglandins, thymol, camphor, extracts or other products of a vegetable or animal origin, e.g. evening primrose oil, starflower oil or currant seed oil, fish oils, cod-liver oil or else ceramides and ceramide-like compounds, etc. It is also advantageous to choose the active ingredients from the group of refatting substances, for example Purcellin oil, Eucerit® and Neocerit®.

The amount of such active ingredients (one or more compounds) in the preparations according to the invention is preferably from 0.001 to 30% by weight, particularly preferably 0.05-20% by weight, in particular 1-10% by weight, based on the total weight of the preparation.

The non-limiting examples below serve to illustrate the present invention.

### EXAMPLE 1

### O/W Cream

| | % by weight |
|---|---|
| Stearic acid | 3.00 |
| Cetyl alcohol | 2.50 |
| Glyceryl stearate | 1.00 |
| Octyl dodecanol | 3.00 |
| Cyclomethicone | 1.00 |
| Myristyl myristate | 2.00 |
| Glycerol | 5.00 |
| Carbomer | 0.10 |
| Sodium hydroxide | q.s. |
| Preservative | q.s. |
| Perfume | q.s. |
| Water, demineralized | ad 100.00 |
| pH adjusted to 6.0-8.0 | |

### EXAMPLE 2

### O/W Lotion

| | % by weight |
|---|---|
| Stearic acid | 2.00 |
| Myristyl alcohol | 0.50 |
| Cetylstearyl alcohol | 0.50 |
| Glycol stearate | 0.25 |
| Glyceryl stearate | 0.75 |
| Cetyl palmitate | 0.70 |
| Octyl palmitate | 4.20 |
| Glycerol | 3.00 |
| Carbomer | 0.10 |
| Magnesium aluminum silicate | 0.20 |
| Potassium hydroxide | q.s. |
| Preservative | q.s. |
| Perfume | q.s. |
| Water, demineralized | ad 100.00 |
| pH adjusted to 6.5-7.5 | |

### EXAMPLE 3

### O/W Lotion

| | % by weight |
|---|---|
| Stearic acid | 2.00 |
| Cetylstearyl alcohol | 1.50 |
| Glyceryl stearate | 1.00 |
| Caprylic/capric triglycerides | 1.00 |
| Cetylstearyl ethylhexanoate | 1.00 |
| Dicaprylyl ether | 2.00 |
| Hydrogenated cocoglyceride | 2.00 |
| Dimethicone | 2.00 |
| Xanthan gum | 0.10 |
| Magnesium silicate | 0.10 |
| Glycerol | 3.00 |
| Perfume, preservative | q.s. |
| Sodium hydroxide | q.s. |
| Dyes, etc. | q.s. |
| Water | ad 100.00 |
| pH adjusted to 7.0-8.0 | |

### EXAMPLE 4

### O/W Emulsion Make-Up

| | % by weight |
|---|---|
| Stearic acid | 1.00 |
| Cetyl alcohol | 2.00 |
| Glycol stearate | 0.50 |
| Glyceryl stearate | 0.25 |
| Cetearyl isononanoate | 2.00 |
| Isopropyl palmitate | 1.00 |
| Dimethicone | 0.60 |
| Cetyl palmitate | 0.70 |
| Glycerol | 3.00 |
| Carbomer | 0.15 |
| Mica | 1.00 |
| Magnesium silicate | 1.00 |
| Iron oxide | 1.00 |
| Titanium dioxide | 2.50 |
| Talc | 5.00 |
| Vitamin A palmitate | 0.10 |
| Sodium hydroxide | q.s. |
| Preservative | q.s. |
| Perfume | q.s. |
| Water, demineralized | ad 100.00 |
| pH adjusted to 6.0-8.0 | |

### EXAMPLE 5

### O/W Cream

| | % by weight |
|---|---|
| Stearic acid | 4.00 |
| Cetyl alcohol | 2.50 |
| Glyceryl distearate | 1.00 |
| Octyldodecanol | 3.00 |
| Hydrogenated coconut fatty acid glyceride | 2.00 |
| Cyclomethicone | 2.00 |
| Vitamin E acetate | 1.00 |
| Xanthan gum | 0.10 |
| Carbomer | 0.10 |
| Retinyl palmitate | 0.20 |
| Glycerol | 3.00 |
| BHT | 0.02 |
| Na₂H₂EDTA | 0.10 |
| Perfume, preservative, dyes | q.s. |
| Potassium hydroxide | q.s. |
| Water | ad 100.00 |
| pH adjusted to 7.0-8.0 | |

### EXAMPLE 6

### O/W Lotion

| | % by weight |
|---|---|
| Stearic acid | 2.00 |
| Cetylstearyl alcohol | 2.00 |
| Glycol stearate | 0.30 |
| Glyceryl stearate | 0.30 |
| Caprylic/capric triglyceride | 1.00 |
| Cetylstearyl ethylhexanoate | 1.00 |
| Dimethicone | 4.00 |
| Myristyl myristate | 1.00 |
| Vitamin E acetate | 2.00 |
| Sodium carbomer | 0.10 |
| Glycerol | 3.00 |
| Perfume, preservative, dyes, etc. | q.s. |
| Sodium hydroxide | q.s. |
| Water | ad 100.00 |
| pH adjusted to 6.0-7.5 | |

### EXAMPLE 7

### Sunscreen Cream

| | % by weight |
|---|---|
| Stearic acid | 3.50 |
| Cetyl alcohol | 1.50 |
| Myristyl alcohol | 1.00 |
| Glyceryl stearates | 1.00 |
| Octyldodecanol | 1.00 |
| C₁₂₋₁₅-alkyl benzoates | 2.00 |
| Paraffin oil | 0.50 |
| Dimethicone | 1.00 |
| Cetyl palmitate | 0.80 |
| Xanthan gum | 0.10 |
| Sodium carbomer | 0.10 |
| Glycerol | 3.00 |
| Octyl methoxycinnamate | 4.00 |
| Benzophenone-3 | 3.00 |
| Octyl salicylate | 3.00 |
| BHT | 0.02 |
| Na₂H₂EDTA | 0.10 |
| Perfume, preservative, dyes, etc. | q.s. |
| Potassium hydroxide | q.s |
| Water | ad 100.00 |
| pH adjusted to 6.5-8.0 | |

That which is claimed:

1. A cosmetic or dermatological preparation in the form of an O/W emulsion comprising:
(I) 1-5% by weight, based on the total weight of the preparation, of one or more neutralized or partially neutralized C₁₄-C₃₂ fatty acids,
wherein the fatty acid is neutralized or partially neutralized with a neutralizing base selected from the group consisting of sodium hydroxide and potassium hydroxide and the neutralizing base is present in an amount to produce a pH range of the preparation of 6.0-8.0 in neutralized or partially neutralized form, and
wherein the content of alkylated ammonium bases is selected to be less than 0.01% by weight;
(II) 0.1 to 1.5% by weight, based on the total weight of the preparation, of one or more compounds selected from the group consisting of monoesters of glycerol, diesters of glycerol, monoesters of propylene glycol, diesters of propylene glycol, monoesters of glycol and diesters of glycol;
(III) 0.5 to 3% by weight, based on the total weight of the preparation, of one or more fatty alcohols selected from the group consisting of branched and unbranched alkyl alcohols having 12 to 40 carbon atoms; and
(IV) 1 to 9% by weight, based on the total weight of the preparation, of a lipid phase composed of
(a) one or more lipids with a melting point below 30° C., and
(b) one or more lipids with a melting point above 30° C.,
wherein the ratio of (a) and (b) is in the range from 2:1 to 6:1,
wherein the lipid phase comprises 0-5% of one or more silicone oils, based on the total weight of the preparation, and
wherein the ratio of (a) to silicone oil is preferably in the range from 5:1 to 1:2.

2. The preparation as claimed in claim 1, wherein the one or more compounds (II) include compounds selected from the group consisting of glyceryl monostearate, glyceryl distearate, propylene glycol monostearate, glyceryl isostearate, glyceryl lanolate, glyceryl myristate, glyceryl laurate, glyceryl oleate, glyceryl stearate citrate.

3. The preparation as claimed in claim 1, wherein the one or more fatty alcohols (II) include fatty alcohols selected from the group consisting of dodecanol (lauryl alcohol, C₁₂H₂₅OH), tetradecanol (myristyl alcohol, C₁₄H₂₉OH), hexadecanol (cetyl alcohol, C₁₆H₃₃OH), octadecanol (stearyl alcohol, C₁₈H₃₇OH), oleyl alcohol (C₁₈H₃₅OH), eicosyl alcohol (C₂₀H₃₉OH), docosanyl alcohol (C₂₂H₄₃OH), tetracosanyl alcohol (carnaubyl alcohol, C₂₄H₄₉OH), hexacosanyl alcohol (ceryl alcohol, C₂₆H₅₃OH), triacontyl alcohol (myricyl alcohol, C₃₀H₆₁OH).

4. The preparation as claimed in claim 1, wherein the one or more fatty acids (I) include fatty acids selected from the group consisting of stearic acid and palmitic acid (stearin).

5. The preparation as claimed in claim 1, wherein the one or more silicone oils in the lipid phase (IV) include monomers having the following structural elements: 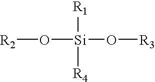
wherein R₁-R₄ are selected from alkyl and aryl radicals.

6. The preparation as claimed in claim 1, wherein the one or more silicone oils in the lipid phase (IV) include linear silicones having two or more siloxyl units having the following structural elements: 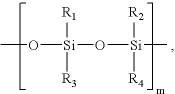
wherein R₁-R₄ are selected from alkyl and aryl radicals and m is from 2-200 000.

7. The preparation as claimed in claim 1, wherein the one or more silicone oils in the lipid phase (IV) include cyclic silicones having the following structural elements: 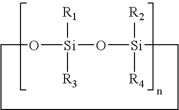
wherein R₁-R₄ are selected from alkyl and aryl radicals and n is from 3/2 to 20.

8. The preparation as claimed in claim 1, wherein the one or more silicone oils in the lipid phase (IV) include silcone oils selected from the group consisting of phenyltrimethicone, dimethicone, phenyldimethicone, cyclomethicone (octamethylcyclotetrasiloxane), hexamethylcyclotrisiloxane, polydimethylsiloxane, poly(methylphenylsiloxane), cetyldimethicone, and behenoxydimethicone.

9. The preparation as claimed in claim 8, wherein the one or more silicone oils in the lipid phase (IV) include phenyltrimethicone.

10. The preparation as claimed in claim 1, wherein the lipid phase (IV) includes mixtures selected from the group consisting of cyclomethicone and isotridecyl isononanoate, and cyclomethicone and 2-ethylhexyl isostearate.

11. The preparation as claimed in claim 1, wherein the lipid phase comprises 0.6-4% of one or more silicone oils, based on the total weight of the preparation.

12. The preparation as claimed in claim 1, wherein the one or more lipids (a) in the lipid phase include one or more esters selected from the group consisting of (i) esters of saturated or unsaturated, branched or unbranched alkanecarboxylic acids having a chain length of from 3 to 30 carbon atoms and saturated or unsaturated, branched or unbranched alcohols having a chain length of from 3 to 30 carbon atoms, and (ii) esters of aromatic carboxylic acids and saturated or unsaturated, branched or unbranched alcohols having a chain length of from 3 to 30 carbon atoms.

13. The preparation as claimed in claim 12, wherein the one or more lipids (a) in the lipid phase include one or more esters selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and jojoba oil esters.

14. The preparation as claimed in claim 1, wherein the lipid phase (IV) inicudes one or more compounds selected from the group consisting of dialkyl ethers, and the group of saturated or unsaturated, branched or unbranched alcohols.

15. The preparation as claimed in claim 1, wherein the lipid phase (IV) includes one or more compounds selected from the group consisting of branched and unbranched hydrocarbons and branched and unbranched hydrocarbon waxes.

16. The preparation as claimed in claim 1, wherein the one or more lipids (b) in the lipid phase include candelilla wax, carnauba wax, japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes, microcrystalline waxes, glyceryl tribehenate, C₁₆₋₃₆ fatty acid triglyceride, montan ester waxes, Sasol waxes, hydrogenated jojoba waxes, synthetic or modified beeswaxes, polyalkylene waxes, polyethylene glycol waxes, hydrogenated vegetable oils, triglycerides, fatty acids, fatty acid esters, and glycol esters, and stearoxytrimethylsilane.

17. The preparation as claimed in claim 1, wherein the one or more lipids (b) in the lipid phase include cetyl palmitate.

18. The preparation as claimed in claim 1, wherein the lipid phase (IV) inlcudes one or more compounds selected from the group consisting of 2-ethylhexyl isostearate, octyidodecanol, isotridecyl isononanoate, isoeicosane, 2-ethylhexyl cocoate, C₁₂₋₁₅ alkyl benzoates, cetylstearyl ethylhexanoate, isopropyl myristate, caprylic/capric triglycerides, and dicaprylyl ethers.

19. The preparation as claimed in claim 1, further comprising (V) one or more alcohols, diols or polyols, and ethers thereof, selected from the group consisting of ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether, isopropanol, 1,2-propanediol and glycerol.

20. A cosmetic or dermatological preparation in the form of an O/W emulsion comprising:
(I) 1-5% by weight, based on the total weight of the preparation, of one or more fatty acids selected from the group consisting of stearic acid and palmitic acid (stearin) that are neutralized or partially neutralized by one or more neutralizing bases selected from the group consisting of sodium hydroxide and potassium hydroxide;
(II) 0.1 to 1.5% by weight, based on the total weight of the preparation, of one or more compounds selected from the group consisting of glyceryl monostearate, glyceryl distearate, propylene glycol monostearate, glyceryl isostearate, glyceryl lanolate, glyceryl myristate, glyceryl laurate, glyceryl oleate and glyceryl stearate citrate;
(III) 0.5 to 3% by weight, based on the total weight of the preparation, of one or more fatty alcohols selected from the group consisting of monohydric alcohols having 12-30 carbon atoms in straight chains;
(IV) one or more lipids with a melting point below 30° C.;
(V) one or more lipids with a melting point above 30° C.;
(VI) greater than 0 to 5% of one or more silicone oils; based on the total weight of the preparation;
wherein (IV), (V) and (VI) comprises 1 to 9% by weight, based on the total weight of the preparation;
wherein the ratio of (IV) to (V) is in the range from 2:1 to 6:1;
wherein the ratio of (IV) to (VI) is preferably in the range from 5:1 to 1:2;
wherein the amount of alkylated ammonium bases is less than 0.01% by weight; based on the total weight of the preparation; and
wherein the pH of the preparation is 6.0-8.0.
